(19) United States
(12) Patent Application Publication
(10) Pub. No.: US 2010/0047768 A1
(43) Pub. Date: Feb. 25, 2010

(54) AMPLIFICATION OF SINGLE VIRAL GENOMES

(75) Inventors: Shannon Williamson, San Diego, CA (US); Lisa Zeigler, San Diego, CA (US); Thomas Ishoey, La Jolla, CA (US)

Correspondence Address: VENABLE LLP, P.O. BOX 34385, WASHINGTON, DC, 20043-9998, US

(73) Assignee: J. Craig Venter Institute, Inc., Rockville, MD (US)

(21) Appl. No.: 12/543,046

(22) Filed: Aug. 18, 2009

Related U.S. Application Data
(60) Provisional application No. 61/136,203, filed on Aug. 18, 2008.
(60) Provisional application No. 61/179,206, filed on May 18, 2009.

(51) Int. Cl. C12Q 1/70; C12P 19/34

(52) U.S. Cl. 435/5; 435/91.1

(57) ABSTRACT

The present invention relates, e.g., to a method for amplifying the genome of a single virus particle from a mixture of virus particles, comprising (a) subjecting the mixture of virus particles to flow cytometry and identifying a sorted sample that putatively contains a single virus particle, (b) imbedding the sorted sample comprising the putative single viral particle in a solid matrix (e.g., low melting agarose); (c) visualizing the embedded virus particle (e.g., by EFM and/or confocal microscopy) to confirm that a single particle is embedded; and (d) exposing the nucleic acid from the visualized, embedded single, discrete viral particle (e.g., by alkali treatment) and amplifying the genomic viral nucleic acid in situ (e.g., by MDA).

This application claims the benefit of the filing dates of U.S. Provisional Application Ser. No. 61/136,203, filed Aug. 18, 2008 and U.S. Provisional Application Ser. No. 61/179,206, filed May 18, 2009, each of which is hereby incorporated by reference in its entirety.

### FIELD OF THE INVENTION

This invention relates, e.g., to methods for isolating and amplifying nucleic acids from single viral particles.

### BACKGROUND INFORMATION

Whole genome amplification and sequencing of single microbial cells has revolutionized the field of microbial ecology by allowing researchers to directly examine the genomic contents of individual cells in the absence of prior cultivation efforts (Binga et al. (2008) Isme J 2, 233-241). Viruses are the most numerous and most diverse biological entities on our planet (Edwards et al. (2005) Nat Rev Microbiol 3, 504-510). They affect every aspect of our lives by shaping the environments that surround us, our immune responses and even our genomes. The field of environmental viral metagenomics has gained momentum over the past five years (Breitbart et al. (2002) Proc Natl Acad Sci USA 99, 14250-14255; Breitbart et al. (2002) Proceedings of the Royal Society of London Series B-Biological Sciences 271, 565-574; Angly et al. (2006) PLoS Biol 4, e368; Culley et al. (2006) Science 312, 1795-1798; Bench et al. (2007) Appl Environ Microbiol 73, 7629-7641; Williamson et al. (2008) PLoS ONE 3, e1456). However, sequencing of individual environmental viral genomes is currently dependent on the establishment of cultivable virus-host systems. Furthermore, the uncertainty of confirming the presence of only one cell during single cell isolation has proven difficult, even for larger cell types. Given that many viral particles are much smaller (25 nm to 100 nm) than the average bacterial cell (0.3-1.5 μm), it is even more difficult to confirm the presence of only a single viral particle. There is a need for a method by which single virions can be isolated and their genomes amplified, for example in preparation for genomic sequencing. The development of such a single virus amplification (SVA) technique could change the paradigms of virology, from ecology to infectious disease.

### DESCRIPTION OF THE DRAWINGS

FIG. 1 illustrates diagrammatically a method of the invention. 1) Viral suspensions are diluted in a suitable buffer (e.g., TE (Tris-EDTA)), 2) subjected to flow cytometry (e.g, using a BD FACSAria II equipped with a Forward Scatter PMT (FSC PMT)), 3) sorted directly onto a suitable receptacle (e.g., PTFE slides with 24 wells, 4) Wells are checked for single viral particles using EFM and/or confocal microscopy, 5) Whole genome amplification (e.g., via MDA method), 6) Characterization of isolated viral particles using a) specific PCR (e.g., multiplex PCR), b) neural networks, and/or c) RAPD PCR.

FIG. 2 shows flow cytometry of bacteriophage standards T4 and lambda.

FIG. 3 illustrates the MDA procedure.

FIG. 4 shows epifluorescent visualization. FIG. 4A shows phage particle localization post-immobilization. FIG. 4B shows the genome of a phage particle, which was amplified using multiple displacement amplification (MDA) of the embedded particle.

FIG. 5 shows multiplex PCR to determine phage suspension content. Gel electrophoresis analysis of optimized PCR. Lane 1: 1 kb plus marker (Invitrogen); Lane 2: lambda integrase (int) gene; Lane 3: T4 gp23 gene encoding the major capsid protein; Lane 4: both lambda int and T4 gp23.

FIG. 6 shows whole genome amplification following single virus isolation. Lane 1: 1 kb plus marker (Invitrogen); Lane 2: Viral isolate 1 (A1); Lane 3: Viral isolate 2 (B2).

FIG. 7 shows Probabilistic Classification of Single Virus

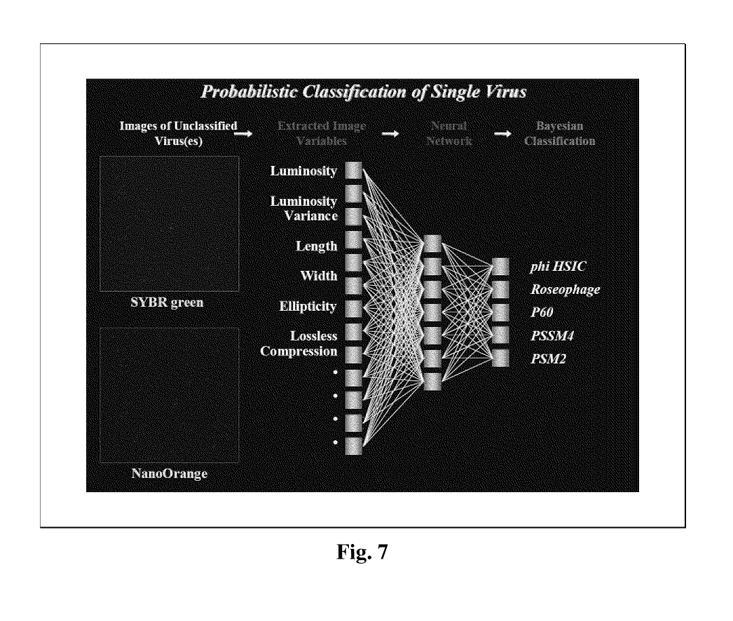

FIG. 8 shows a PTFE slide, without the addition of a sample (FIG. 8A) or with the addition of sample (e.g., the addition of the agarose used for immobilization of viral particles) (FIG. 8B).

FIG. 9 shows confocal microscopy of single marine virus isolated.

FIG. 10 shows amplified genomic material of single viruses isolated from marine virioplankton. Lane 1: 1 kb plus marker (Invitrogen); Lane 2: no template control; Lane 3: Isolate GS260_B1; Lane 4: Isolate GS260_B3; Lane 5: Isolate GS260_B4.

### DESCRIPTION OF THE INVENTION

The present invention relates, e.g., to a method for isolating and amplifying single viral particles from a mixture of viral particles, without cultivating the viruses in vivo (passaging them in a host cell).

The inventors have found, surprisingly, that one can isolate single virus particles from a mixture of virus particles with a two-step procedure: (1) sorting the mixture by flow cytometry to generate sorted samples which putatively contain single virus particles; and (2) embedding (immobilizing) the sorted samples of the sorted viruses which putatively contain single virus particles in a matrix, such as low melting agarose, in order to unequivocally isolate individual viral particles. By using a method such as epifluorescent microscopy (EFM) and/or confocal microscopy to visualize the embedded particles, an investigator can determine the position of any individual particle, and/or can confirm that a single particle is, in fact, present. The inventors have also shown that one can amplify the nucleic acid of the individual, isolated embedded viral particles in situ (in the presence of the agarose).

Advantages of a method of the invention include that, by eliminating the requirement that viruses be propagated/cultivated in host organisms, one can eliminate the potential selection bias that is inherent to the cultivation process, and one can save the time and money required for carrying out the cultivation. A method of the invention is particularly useful for studying viral species for which suitable hosts and/or cultivation requirements are unknown (e.g., environmental or clinical samples which contain unknown or uncharacterized viruses). Another advantage of a method of the invention is that, by being able to visualize the embedded viral particles before sequencing their nucleic acid, one can insure that the nucleic acid from only a single viral particle is being sequenced. Another advantage is that embodiments of the invention (e.g., embodiments in which laser capture microdissection (LCM) is employed for the isolation of amplified viral genomes from a gel matrix, and sophisticated automatic sequencing systems are used) can be readily adapted for high throughput analysis.

One aspect of the invention is a method for amplifying the genome of a single virus particle from a mixture of virus particles. The method comprises the following steps, which are also discussed in more detail in the Examples herein:

1. The mixture of virus particles is subjected to flow cytometry, to sort the virus and to generate sorted samples which putatively contain single virus particles. In one embodiment of the invention, the Flow Cytometer is a high-resolution device equipped with a Side Scatter PMT (SSC PMT) Forward Scatter PMT (FSC PMT). The FSC PMT is a highly sensitive photodetector called a photomultiplier tube (PMT) that collects light scattered in the side and forward direction. A PMT is used to amplify weak signals, such as those that would be generated from viral particles.

2. Sorted samples containing approximately one viral particle are embedded in a solid matrix. Any suitable matrix material can be used, e.g., low melting agarose (which is sometimes referred to herein as LMP agarose, or low melt agarose). A variety of forms of agarose, as well as other matrix materials, can be used. Much of the discussion herein is directed to the use of low melt agarose. It will be evident to a skilled worker that this discussion encompasses the use of other suitable matrices, as well.

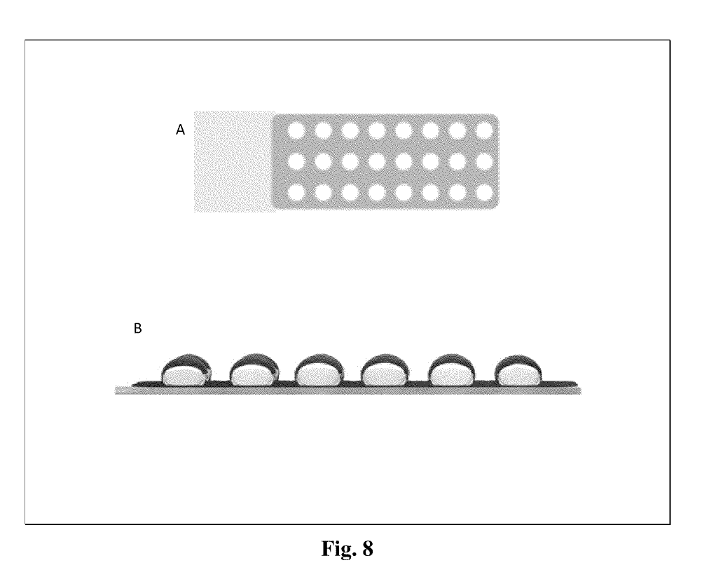

In one embodiment of the invention, a sorted sample which putatively contains a single virus particle is placed directly in a suitable small vessel that contains low melt agarose. For example, the sorted sample can be placed in a well of a polytetrafluoroethylene (PTFE) slide into which low melt agarose has been added. PTFE slides are extremely hydrophobic slides that can be used for controlling cross-contamination between the wells. They are available, e.g., from Electron Microscopy Sciences. See FIG. 8 for an illustration of a PTFE slide. In one embodiment of the invention, about 5 ul of low melt agarose, at about 37° C., is applied to the wells of a PTFE slide with 24 wells and allowed to solidify. A sorted sample, in a sub-nanoliter volume, is added to each well and allowed to sink into the agarose; and about 5 ul of molten low melt agarose, at about 37° C., is added to the well and allowed to solidify, to create a “bead” of agarose comprising the putative single virus particle. In this manner, the virus is embedded in (immobilized in) the agarose. In another embodiment, the sorted sample is placed in a well of a 96-well plate into which low melt agarose has been added, and the sample is embedded as above.

In another embodiment of the invention, a thin layer of low melt agarose is first applied to a (flat) glass microscope slide and allowed to solidify. Sorted samples that putatively contain a single virus particle are collected onto the slides. If desired, the sorted samples can be collected onto defined positions onto the thin layer of solidified agarose, for example in a grid pattern with, e.g., 1-6 samples per slide, to facilitate the localization of the samples. The sorted samples are allowed to penetrate (sink into) the agarose; and the surface is then covered with another thin layer of molten agarose. This is another way by which a virus particle can be embedded in low melt agarose.

3. In order to insure that each sorted sample contains only a single virus particle, the following procedure can be carried out. The embedded virus particles are visualized, for example by EFM and/or confocal microscopy, and are enumerated (counted). In some cases, confocal microscopy, alone, is sufficient to determine if a single particle is present. For visualization with EFM, the particles can be stained with suitable dyes, for example a fluorescent dye that binds to and/or intercalates into the nucleic acid of the virus, such as SYBER Gold or SYBR Green (both from Invitrogen). Other suitable dyes will be evident to a skilled worker and include, e.g., 4′, 6-diamidino-2-phenylindole dihydrochloride, and others. However, it is not necessary to stain the particles when using an epifluorescence microscope. Rather, a 375 μM “cube set” for the EFM microscope can be used. Certain proteins that are present in viral particle capsids auto-fluoresce at this wavelength, and thus can be visualized in the absence of staining. Another method for visualization is to use confocal microscopy. In this procedure, the particles are stained with a suitable stain, many of which will be evident to a skilled worker (e.g., the nucleic acid stains, SYBR Gold/Green, or the protein stain, NanoOrange), and visualized/enumerated with a confocal microscope. Confocal microscopy has the advantage that, by taking into account the three-dimensional imaging, one can readily determine if a single virus particle is present in a particular defined area, or if two of more virus particles are stacked above one another.

4. The nucleic acid of single, discrete virus particles is exposed by a conventional procedure (e.g. by alkaline lysis, heat or KOH solution), and the exposed nucleic acid is amplified in situ (from within a solid matrix, such as an agarose matrix) by a whole genome amplification procedure.

In one embodiment of the invention, virus particles are sorted onto PTFE slides containing low melt agarose, in order to form agarose “beads,” and the embedded particles are visualized. A bead can then be selected which contains a single virus particle, and the nucleic acid exposed and amplified from within the agarose. Alternatively, a bead can be selected which contains more than one virus particle, e.g. as many as about 10 particles. The individual, visualized virus particles can be etched out and catapulted into new containers by laser capture microdissection (LCM), and the nucleic acid, still embedded in agarose, exposed and amplified. The new containers can be, e.g., microfuge tubes or microtiter plates. In another embodiment of the invention, sorted samples are collected onto discrete, addressable regions of a (flat) microscope slide, and the nucleic acid is exposed and amplified directly on the slide if the viral particles are well enough separated from one another. Alternatively, if the position of a viral particle is known, the agarose containing the virus can be excised (e.g. with a sterile razor, or by LCM) and removed to a suitable vessel, where the nucleic acid, still embedded in agarose, is then exposed and amplified.

A number of methods have been developed for exponential amplification of small amounts of nucleic acids, which can be performed in situ (in a background of a matrix, such as low melt agarose). These include a variety of methods of whole genome amplification (WGA), e.g., the isothermal amplification method, multiple displacement amplification (MDA). In one form of this method, two sets of primers are used that are complementary to opposite strands of nucleotide sequences flanking a target sequence. Amplification proceeds by replication initiated at each primer and continuing through the target nucleic acid sequence, with the growing strands encountering and displacing previously replicated strands. In another form of the method, a random set of primers is used to randomly prime a sample of genomic nucleic acid. The primers in the set are collectively, and randomly, complementary to nucleic acid sequences distributed throughout nucleic acid in the sample. Amplification proceeds by replication initiating at each primer and continuing so that the growing strands encounter and displace adjacent replicated strands. MDA is illustrated in the Examples herein.

Other suitable methods of whole genome amplification of small amounts of nucleic acid, which can be carried out in situ, include, e.g., ligation-mediated PCR (LMP PCR) (Tanabe et al. (2003) Genes, Chromosomes and Cancer 38, 168-176), such as OmniPlex technology (Rubicon, Inc.), which takes fragmented genomic DNA (4-5 ng) followed by ligation of universal adapters and then amplifies using universal primers (Langmore, J P. (2002) Pharmacogenomics 3, 557-560); degenerate oligonucleotide primed PCR (DOP-PCR), which uses random primers to amplify, via PCR, genomic DNA (Telenius et al. (1992) Genomics 13, 718-725; and T7-based linear amplification of DNA (TLAD), in which a polyT tail is added to the 3′ end of fragmented genomic DNA, which then provides a binding site for a T7 promoter with a poly A tail at the 3′ end, and second strand synthesis is then performed followed by in vitro transcription using T7 polymerase in an isothermal reaction (Liu et al. (2008) Cold Spring Harbor Protocols). The method illustrated in the Examples herein is MDA, but it is to be understood that other suitable methods of WGA can also be used.

Subsequent to initial amplification by a WGA method (e.g., about 10-20, for example about 15, minutes of amplification), one can also employ additional amplification methods in which the enzymes are not as processive, such as the polymerase chain reaction (PCR), ligase chain reaction (LCR), self-sustained sequence replication (SSR), nucleic acid sequence based amplification (NASBA), strand displacement amplification (SDA), and amplification with Qβ replicase (see, e.g., Birkenmeyer et al. (1991) J. Virological Methods 35, 117-126 and Landegren (1993) Trends Genetics 9, 199-202).

Following in situ amplification of the nucleic acid, the amplified nucleic acid can be visualized (e.g. by EFM), if necessary, excised (e.g. by physical dissection), separated from the agarose by treating with agarase, and purified with a conventional phenol/chloroform/ethanol procedure.

It is often desirable to confirm that amplified nucleic acid is from a single virus particle and/or that it contains one or more viral nucleic acid sequences of interest. Procedures for performing such a confirmation (e.g. by performing specific PCR, or Neural network training) are discussed in the Examples. Other suitable methods, such as Field Inversion Gel Electrophoresis (FIGE) can also be employed. This method is discussed, e.g., in Birkenmeyer et al. (1991) J. Virological Methods 35, 117-126 and Landegren (1993) Trends Genetics 9, 199-202).

In initial studies to show that a single virus particle could be isolated from a mixture of virus particles and amplified in situ by a whole genome amplification procedure, virus samples were not subjected to flow cytometry. Rather, the samples were first subjected to dilution, such that only a few virus particles were expected to be in each diluted sample, enumerated in order to identify samples that putatively contained single particles, and samples that putatively contained a single virus particle were then embedded into agarose and amplified by a method of WGA. This method is illustrated in Example I.

Briefly, an aliquot of a diluted sample containing virus particles is applied to a filter which retains the particles (e.g., one can apply about 25 μl of solution to a 0.2 μm (micron) filter); and the virus is stained with a fluorescent dye, as discussed above. The stained particles are visualized by EFM and are enumerated. Based on the enumeration, dilutions (e.g., serial dilutions) are carried out, if needed, with the virus particles in at least one of the enumerated, sorted aliquots, to generate aliquots containing approximately one virus particle. This procedure is sometimes referred to herein as limit dilution or end-point dilution. Methods for carrying out dilutions (e.g., serial dilutions) are conventional and will be evident to a skilled worker. They can be a series of, e.g., 10-fold dilutions, 5-fold dilutions, or dilutions of other amounts, which can be the same or different, into a suitable liquid, such as a buffer or culture medium, in which the viruses are stable, such that, in the highest dilution, approximately one homogeneous (pure) virus particle is present. A dilution which putatively contains a single virus particle is then spotted onto a slide to which has been applied a thin layer of low melt agarose, e.g. in an aliquot of abut 200 nl. To facilitate the localization of each spot, it is useful to spot the aliquots in defined positions, for example in a grid pattern with, e.g., 1-6 samples per slide. The samples are then embedded, visualized if necessary to confirm that a single virus particle is present, the nucleic acid is exposed, and the nucleic is amplified as described above. Alternatively, the diluted samples which putatively contain a single virus particle are placed into small receptacles containing agarose, such as the 96-well plates or PTFE plates as described above, visualized if necessary to confirm that a single virus particle is present, and nucleic acid in the embedded samples is exposed, and the nucleic is amplified as described above. This method can be used as an alternative to the methods employing flow cytometry.

Amplified nucleic acid from a single virus particle can be analyzed further by any of a variety of procedures, including, e.g., hybridization, haplotyping, microsatellite analysis, restriction enzyme analysis, RAPD PCR or a variety of SNP typing techniques. In one embodiment of the invention, the amplified nucleic acid is sequenced, using any of a variety of conventional procedures. High throughput sequencing procedures, used in conjunction with the isolation and amplification methods of the invention, can be particularly powerful for sequencing large numbers of viral species in, e.g., an environmental sample.

In one embodiment of the invention, the in situ amplified nucleic acid is visualized and removed from an agarose layer by laser capture dissection (LCM), a conventional method for which devices are commercially available. The excised agarose can be transferred (e.g., catapulted) into individual microfuge tubes or microtiter plates and analyzed (e.g. amplified and sequenced) directly. Because both the LCM and sequencing procedures are highly automated, this entire procedure can be adapted to a high throughput procedure (automated and/or performed robotically).

Methods of the invention can be adapted for a variety of uses. For example, methods of the invention allow one to generate extensive viral reference genome libraries, providing a first step toward constraining the amazing level of viral genotypic diversity witnessed within environmental samples to date. Single viral particle analyses will also compliment current viral metagenomic efforts by helping to guide assembly strategies and enabling comparative genomic analyses through fragment recruitment (Rusch et al. (2007) PLoS Biol 5, e77).

In one embodiment of the invention, a mixture of virus particles from a natural population of viruses in an environmental sample (e.g., virioplankton communities from aquatic environments and viral communities from soil, sediments, biofilms, the deep subsurface and aerosols) is analyzed by a method of the invention. Some typical procedures for preparing and analyzing such samples are discussed in the Examples. An investigator can then characterize the genotypes of viruses present in an environmental sample by amplifying the nucleic acid of single particles of the viruses (generally a viral concentrate) by a method of the invention and further characterizing the amplified nucleic acid, for example by sequencing it.

Another embodiment of the invention is a method for mining for useful genes in a sample containing a mixture of viruses (e.g., from clinical or environmental samples), comprising amplifying the nucleic acid of single particles of the viruses by a method of the invention, followed by sequencing, and identifying novel viral sequences and/or genes of interest from the amplified nucleic acid. Genes can be identified using conventional bioinformatic methods that provide ORF calling, taxonomic classification and functional characterization.

A viral (virus) “particle,” as used herein, refers to a discrete entity in which a protein, an envelope, or both encapsulate the genomic nucleic acid. The genomic nucleic acid can be DNA (single-stranded, double-stranded, or a combination thereof) or RNA (single-stranded, double-stranded), and it can be in a linear form or fragmented (segmented). Art-recognized methods are available for amplifying either single-stranded or double-stranded DNA; and RNA genomes can be converted to single-stranded or double-stranded DNA by conventional procedures, for example, by producing a cDNA molecule of the RNA. Conventional methods for accomplishing this or other molecular biology techniques mentioned herein are described, e.g., Sambrook et al., Molecular Cloning. A Laboratory Manual, current edition, Cold Spring Harbor Laboratory Press, Cold Spring Harbor, N.Y. or Ausubel et al., Current Protocols in Molecular Biology, John Wiley & sons, New York, N.Y.

Any combination of the materials useful in the disclosed methods can be packaged together as a kit for performing any of the disclosed methods. A skilled worker will recognize components of kits suitable for carrying out any of the methods of the invention. For example, a kit for amplifying the genome from a single virus particle from a mixture of virus particles can comprise any combination of reagents for conducting flow cytometry, and/or reagents for embedding sorted samples containing single viral particles in a matrix such as low melting agarose, and/or reagents for amplifying embedded nucleic acid in situ by MDA, etc. Optionally, a kit of the invention comprises instructions for performing the method. Other optional elements of a kit of the invention include suitable buffers, or the like, containers, or packaging materials. The reagents of the kit may be in containers in which the reagents are stable, e.g., in lyophilized form or stabilized liquids. The reagents may also be in single use form, e.g., in a form for a single amplification.

In the foregoing and in the following examples, all temperatures are set forth in uncorrected degrees Celsius; and, unless otherwise indicated, all parts and percentages are by weight.

### EXAMPLES

### Example I

### Material and Methods Using Serial Dilution Procedures for Single Viral Particle Isolation and Amplification

### 1. Bacteriophage Propagation:

For the proof-of-concept study with bacteriophage T4 and lambda which is presented in Examples I-IV, bacteriophage were propagated as follows: Bacteriophage standards of T4 (ATCC 11303-B4) and lambda (ATCC 23274-B2) and the Escherichia coli host for each (ATCC 11303; ATCC 23274, respectively) were propagated in liquid culture. Cultures were centrifuged at 1000 rpm for 20 minutes and supernatant collected, following filtration through a 0.22 μm Pall filter.

For the characterization of environmental samples, viral propagation is not carried out. Rather, the virus is concentrated and purified directly from the environmental sample.

2. Phage Detection and Sorting with Flow Cytometry:

T4 and Lambda Phage were mixed and stained with 0.5 uL/mL SYTO BC stain (Invitrogen) and sorted on a BD FACSAria II I Flow Cytometer equipped with a custom Forward Scatter PMT (FSC PMT). Threshold values of 1000 (FSC PMT) and 200 (SSC) were set to maximize S:N ratios. Stained sample mixtures were detected with an 89% efficiency and sorted into a 96-well plate with 1, 10, 50, 100 and 500 viral particles per well.

### 3. Thin-Layer Agarose Immobilization

30 μl of molten 1% low-melting point (LMP) agarose was applied in a thin layer to a standard microscope slide. A cover slip (25 mm) was placed on top of the gel prior to solidification and the gel was allowed to cool for 30 minutes. The slide was placed on a grid, and the cover slip was carefully removed in order to apply a plotted array of viral dilutions (200 nl per spot). After 20 minutes, the samples were overlaid with another 30 ∞l of cooled (42° C.) molten 0.5% LMP agarose and the cover slip reapplied.

### 4. Epifluorescent Microscopy

Epifluorescence direct counting was performed for enumeration of flow cytometry suspensions, immobilized phage particles, and to verify genome amplification. All samples were stained with SYBR gold (Invitrogen) and visualized at 100× (Zeiss).

### 5. MDA (Multiple Displacement Amplification)

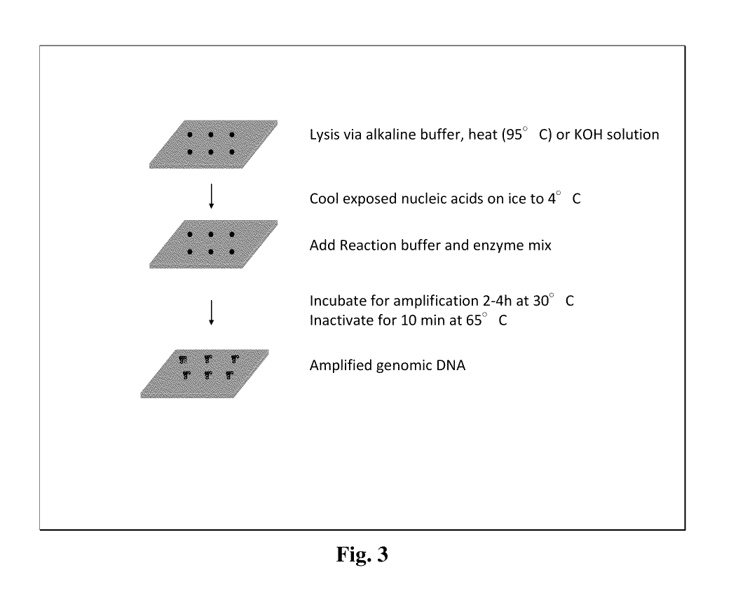

This procedure is illustrated diagrammatically in FIG. 3.

Alkali denaturing solution was added to the gel and incubated on ice for 30 minutes, in order to expose the viral DNA. Following this lysis, neutralization buffer was added to the gel and incubated on ice for 30 minutes.

The exposed DNA was then amplified, in situ, through multiple displacement amplification (MDA) (Dean et al. (2001) Genome Res 11, 1095-1099), utilizing the isothermal Phi29 DNA polymerase. MDA reactions were prepared via the Repli-g kit (Qiagen) according to the manufacturer's instructions, with the modification that the denaturing solution was incubated for 30 minutes, and the master mix final volume was increased to 30 μl per agarose layer. The cover slip was removed and replaced between each step.

### 6. Multiplex PCR

The glass slide was placed on a grid that corresponded with the plotted array of diluted bacteriophage particles, behaving as a guide to where the phage particles were originally deposited. The gel was subsequently dissected on the slide in order to separate the distinct dilutions prior to PCR validation. Phage DNA was purified from the gel slices in microcentrifuge tubes using a β-agarase reaction followed by standard phenol/chloroform extraction and ethanol precipitation.

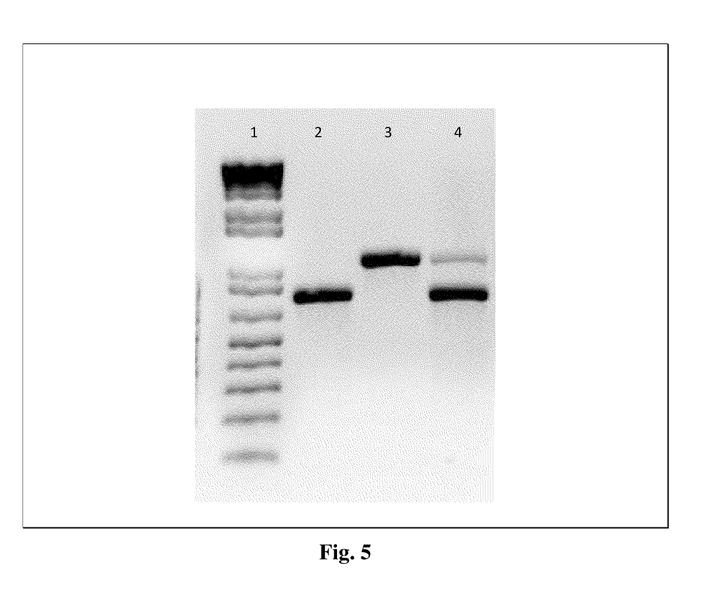

T4-specific primers were designed for the major capsid protein (gp23 gene) of bacteriophage T4, and for the lambda integrase gene (int) for bacteriophage lambda, with the expected band sizes of PCR amplicons of 1050 bp and 750 bp, respectively. The PCR primers for each bacteriophage are shown in FIG. 5. Standard PCR reaction conditions were used, with an annealing temperature of 56° C. (based on gradient PCR optimization of multiplex PCR).

### Example II

### Material and Methods for Flow-Cytometry and PTFE Slide Use in Isolation and Amplification of Single Viral Particles

### 1. Flow Cytometry Parameters

Viral particle suspensions are sorted on a BD FACSAria II Flow Cytometer equipped with a custom Forward Scatter PMT (FSC PMT). The particles were diluted in TE (Tris-EDTA, pH 7.2, Invitrogen) to an appropriate titer for an event rate of 200 events s-1. Any suitable buffer can be used: in this experiment, TE was used because it improves the emission signal of stained viruses (C. Brussaard (2004) Applied and Environmental Microbiology 70, 1506-1513. Thresholds were set to FSC (forward scatter) at 1000 and SSC (side scatter) at 200 for the T4/lambda particles. The FSC threshold is dropped to 200 when sorting environmental viral concentrates to increase sensitivity of detection. Prior to beginning sort, blanks containing 0.2 um-filtered TE are measured for background recognition. In addition to blanks, unstained and stained viral particles of the sample are measured to a total of 5000 events each. Readings are measured on bi-exponential plots, these consist of the lower scale being linear and the higher being exponential. Viral particle suspensions are stained with SYBR Green I (Invitrogen) and sorted onto PTFE hydrophobic slides (Electron Microscopy Sciences).

### 2. Agarose Immobilization

PTFE slides have 24 spots that are used for agarose immobilization; these spots are sometimes further referred to herein as wells. To each well, 5 ul of low melting point (LMP) agarose, cooled to 37° C., is added. The viral particle suspensions are subsequently sorted onto the LMP agarose droplets in concentrations of 10 events or 1 event. An event is the occurrence of a fluorescently labeled particle. Each well is then overlaid with 5 ul of LMP agarose, cooled to 37° C.

### 3. Visualization and Whole Genome Amplification

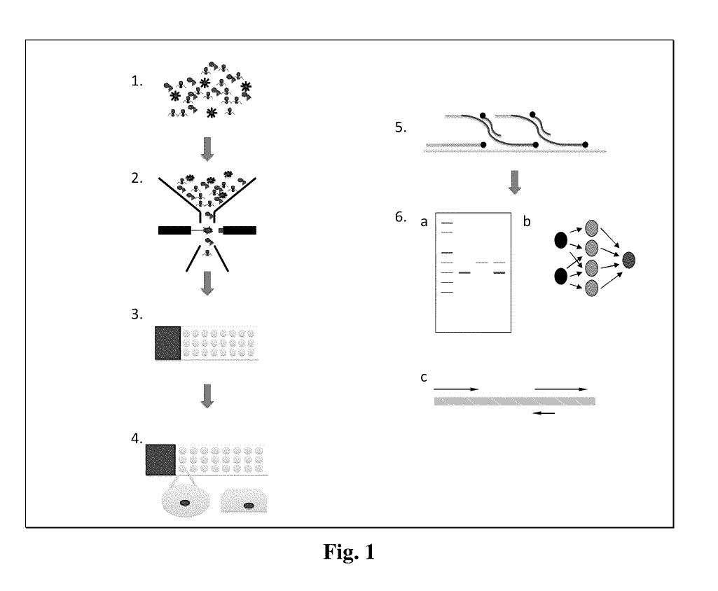

Each imbedded virion(s) (virus(s)) is visualized on the slide using epifluorescence and confocal microscopy to prove that a single viral particle is present in each well (FIG. 1). Once a well is identified to obtain a virion with a 360° view (obtained through confocal microscopy), the viral particles are lysed and their genomic material amplified using the phi29 DNA polymerase and multiple displacement amplification (MDA) reaction (GenomiPhi kit, GE Healthcare). Using this step for lysis and amplification the virions remain immobilized within the agarose droplets. Virions may also be isolated from the agarose plugs using β-agarase, purified, and then their genomic material can be lysed and amplified as above (GenomiPhi kit, GE Healthcare).

### 4. Multiplex PCR

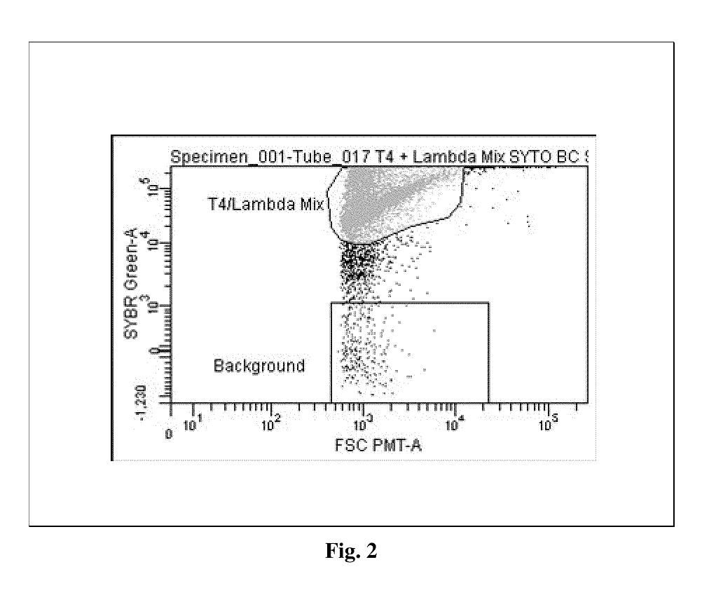

Multiplex PCR used for validation of bacteriophage isolation and type needed optimization prior to being applied on samples. Primer sets specific to gene(s) in each bacteriophage were mixed and used in gradient PCR to identify the annealing temperature for subsequent reactions. Amplification of all products was best at 56° C., with lambda products at the same intensity as individual reaction, FIG. 2a and the T4 gp23 gene product dropping approximately 10-fold, FIG. 2b.

### Example III

### Flow Cytometry, Followed by Immobilization of a Limit Dilution of a Mixture of T4 and Lambda Bacteriophages, and In Situ Amplification of their Genomic DNA by MDA

1. Flow cytometry: A high titer viral concentrate—a mixture of the two well-characterized E. coli bacteriophages, T4 and lambda, both of whose genomic sequences have been determined—was sorted by flow cytometry into 96 well plates, receiving a range of particles ranging from about 500, 100, 50, 10 and 1. Each well containing sorted particles received 100 μl of sterilized water. FIG. 2 shows flow cytometry of the T4 and lambda bacteriophage standards.

2. 25 ul aliquots of the sorted particles were applied to a 0.2 um filter; the nucleic acid was stained with SYBR Gold; and the particles were enumerated using epifluorescent microscopy. Sorted viral suspensions were further diluted to theoretically one particle based on the EFM counts.

3. Serial dilutions of viral suspensions (e.g., 10³, 10², 10, 1) were embedded in a thin layer of agarose applied to a microscope slide (200 nl volumes/spot). There were typically 4-6 embedded dilutions per slide, arrayed in a grid configuration.

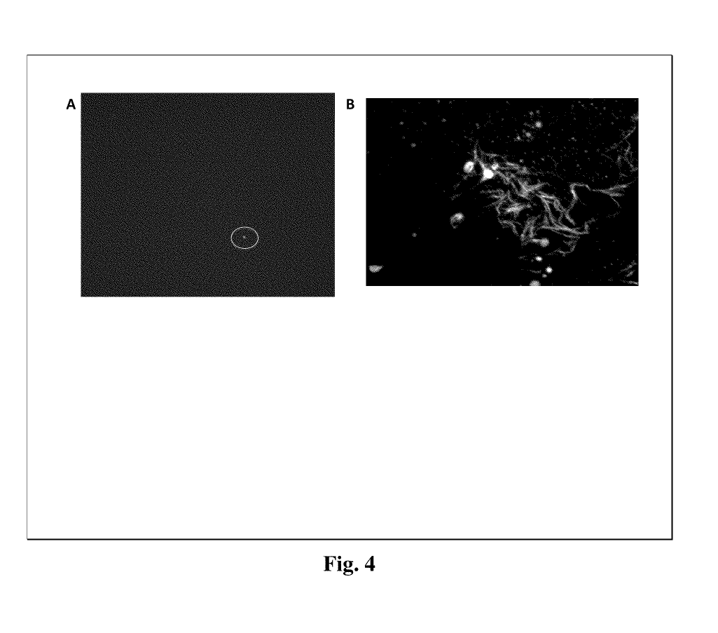

4. The embedded particles were stained with SYBR Gold, visualized and enumerated using EFM. In some experiments, a duplicate slide was made, stained with the protein stain NanoOrange, and visualized/enumerated using confocal microscopy. FIG. 4A shows a typical visualization of phage particles after immobilization.

5. The embedded particles were physically dissected from the agarose layer; the agarose was dissolved (agarase reaction); and viral particles were further diluted. The new dilutions were embedded once again and step 4 was repeated (generally between 1 and 10 times) until we were confident that we had discrete viral particles within each “spot”.

6. When we were convinced that we had captured individual particles, viral nucleic acid was amplified in agarose using MDA (as illustrated diagrammatically in FIG. 3) and the amplified DNA was visualized using EFM. FIG. 4B shows the visualization of the MDA amplified genome of an embedded particle. The visualized, amplified genomes were dissected from the agarose; the agarose was dissolved; and the nucleic acid (starting with dsDNA) was purified through phenol/chloroform extraction.

### Example IV

### Single Virus Isolation

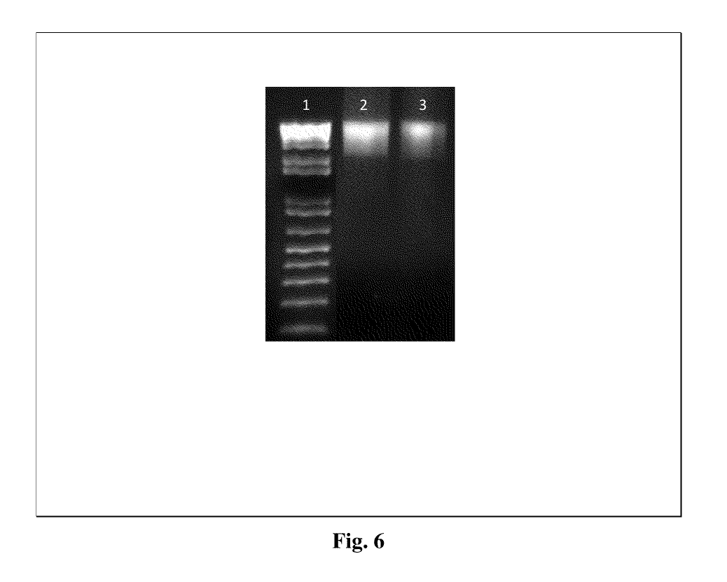

MDA reactions of serially diluted samples post flow sorting and epifluorescent enumeration with subsequent multiplex PCR are shown in FIG. 6. According to our calculations, we have successfully isolated and amplified a single bacteriophage lambda particle.

The results confirmed that the amplified nucleic acid is, in fact, from a single viral particle. The validation step was carried out using pyrosequencing, 454-Titanium sequencing. However, Sanger sequencing or any next generation platform could also be optimized for use. The average JTC 454-Titanium sequence read has >400 high quality bases and 85% of JTC sequencing reactions produce useful reads. It is known that MDA can potentially result in uneven coverage across different areas of a genome. Sequencing reads were assembled using the Celera Assembler, MUMMER and CLC for reference assembly. Annotation of phage genomes and fragment recruitment to the existing lambda genome sequence allowed us to determine that the entire genomes of these phage standards were represented.

### Example V

### Application to Natural Populations of Viruses in Environmental Samples

Samples of natural populations of viruses from an environmental sample are generated as follows:

For aquatic samples, a concentrated virus preparation is separated from contaminants, such as cellular contaminants, by conventional procedures, such as centrifugation through a suitable filter or centrifugation to remove the larger contaminants. The preparation is treated with an appropriate DNase enzyme (e.g., DNAse I) to remove free cellular DNA and is pelleted through a sucrose cushion. Ten unique dsDNA viruses are isolated and amplified by a method of the invention. The isolation and whole in situ genome amplification by MDA are performed following steps 1-6 of Example II.

For more complicated matrices, such as soils or sediments, rather than filtering a sample, the sample can be subjected to three rounds of sonication to remove viral particles from soil/sediment particles, then centrifuged to pellet bacterial cells and other contaminants. The viruses remain in the supernatant and can be concentrated and purified, then treated as above.

If a complex sample, such as a sample of virioplankton, is subjected to flow cytometry in order to isolate individual particles, a method is used as described above, except the flow cytometry thresholds are adjusted to accommodate the varying particle sizes (e.g., virioplankton particle size) known to be present in natural assemblages.

To confirm the presence of individual viruses, we cannot use the procedure described in Example IV, because there are no universal genes in viruses, so we cannot use specific PCR of gene markers to confirm the presence of individual viruses. Instead, we will implement alternative methods to specific PCR in order to determine if individually captured particles are unique. Examples of methods that may be performed are outlined below.

A. RAPD-PCR (Randomly Amplified Polymorphic DNA-PCR) will be used to create molecular fingerprints of the amplified viral nucleic acid from environmental samples (Winget et al. (2008) Applied and Environmental Microbiology 74(9) 2612-2618). Random ten-mer primers will be used to produce molecular fingerprints for each individual amplified marine virus. Since each marine viral “species” should demonstrate a distinctive banding pattern, we will be able to target unique members of the virioplankton community for whole genome sequencing. Unique fingerprints will flag unique viruses for sequencing. This is expected to prevent us from sequencing the same environmental isolate multiple times.

B. Neural network training. This is a technique that is based on the field of astronomy. We capture the light curve data from each viral particle that is visualized through EFM. We have determined that different viral particles (e.g. T4 and lambda) display different fluorescent surface plots. Bimodal curves indicate two particles stuck together. Unimodal curves indicate one particle. Unimodal curves are unique to different viral genotypes. We can capture the numerical data associated with these light curves through a program called ImageJ (from NIH). ImageJ stands for image processing and analysis in Java. A neural network is trained on the numerical data associated with each light curve and the outcome of the neural network allows us to say that a particular particle is either T4 or lambda with 99.9% certainty. With respect to environmental viral isolates, we will obtain light curve data from each individual particle and develop a “neural network library.” As more particles are obtained, we will check them against the growing library to determine if viral particles are unique and should therefore proceed to sequencing.

C. RFLP (Restriction Fragment Length Polymorphism). This is a conventional procedure that is performed on DNA. A RFLP is a variation in the DNA sequence of a genome that can be detected by breaking the DNA into pieces with restriction enzymes and analyzing the size of the resulting fragments by gel electrophoresis. It is the sequence that makes DNA from different sources different, and RFLP analysis is a technique that can identify some differences in sequence (when they occur at a restriction site). This DNA fingerprinting tool allows the classification of single viral genomes based on the banding pattern seen following gel electrophoresis, or better characterized as the genomic variability inherent to that specific virion. If needed, these bands are isolated from the gel using standard procedures and sequenced to identify unique or similar bands between viral genomes.

Following the isolation of the first ten individual viruses, confirmation that single virions have been isolated, and amplification of their genomes, we will establish a marine virus reference genome library, using a 454 pyrosequencing procedure. The initial validation of our methods using Sanger sequencing approaches will allow us to take advantage of the ultra high-throughput and cost-effective nature of 454 pyrosequencing technology. By adopting a barcoding approach, we can achieve ˜180× coverage of ten individual, unique, marine viruses (˜50 kb) in one half 454 run. 454 sequences will be assembled with the Newbler Assembler. We will aggressively identify genes by selecting all open reading frames that are longer than 30 amino acids. BLAST-based clustering approaches will be used to group identified ORFs with known proteins. Functional annotation of identified ORFs will be carried out through comparisons to protein family-based databases.

### Example VI

### Application to Marine Virioplankton Assemblages

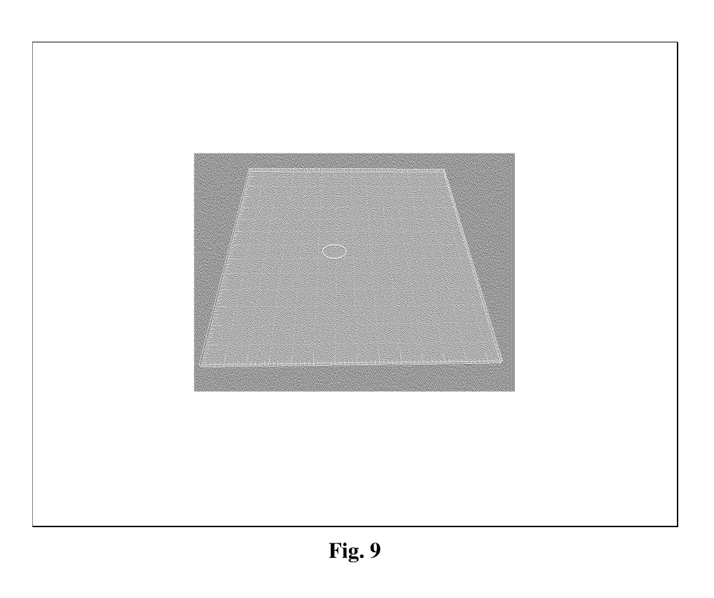

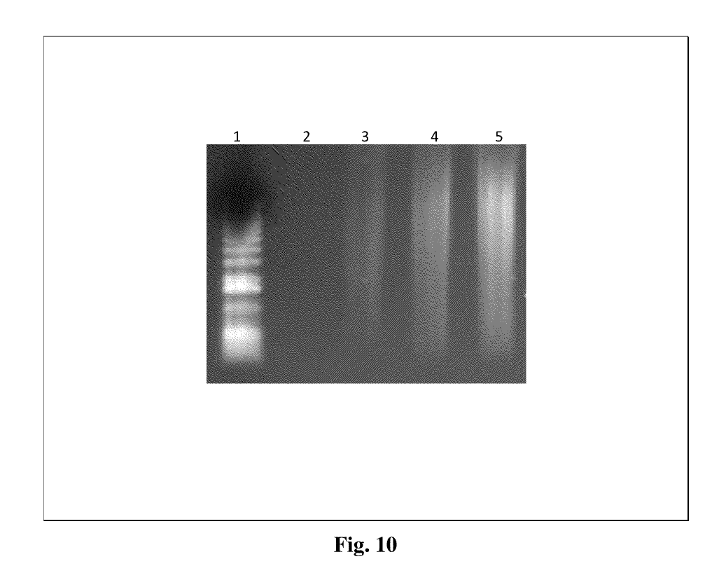

A concentrated virus preparation taken from the California Current was separated from contaminants, such as cellular contaminants, by conventional procedures, such as centrifugation through a suitable filter or centrifugation to remove the larger contaminants. The preparation was treated with an appropriate DNase enzyme (e.g., DNAse I) to remove free cellular DNA and was pelleted via ultra centrifugation through a sucrose cushion. dsDNA virioplankton are isolated through immobilization in agarose on PTFE slides. Confocal microscopy was used to identify wells on the slides that contained a single viral particle (FIG. 9). Genomic DNA was amplified in situ by a method of the invention (FIG. 10). The isolation and whole in situ genome amplification by MDA are performed following steps 1-4 of Example II. Viral genomic DNA was prepared through library construction and is being sequenced by 454 Titanium sequencing.

From the foregoing description, one skilled in the art can easily ascertain the essential characteristics of this invention, and without departing from the spirit and scope thereof, can make changes and modifications of the invention to adapt it to various usage and conditions and to utilize the present invention to its fullest extent. The preceding preferred specific embodiments are to be construed as merely illustrative, and not limiting of the scope of the invention in any way whatsoever. The entire disclosure of all applications, patents, and publications cited above (including U.S. Provisional Applications Ser. No. 61/136,203, filed Aug. 18, 2008 and Ser. No. 61/179,206, filed May 18, 2009) and in the figures, are hereby incorporated in their entirety by reference.

We claim:

1. A method for amplifying the genome of a single virus particle from a mixture of virus particles, comprising
a) subjecting the mixture of virus particles to flow cytometry, thereby generating a sorted sample which putatively contains a single virus particle;
b) embedding the sorted sample containing the putative single virus particle in a solid matrix;
c) visualizing the embedded virus particle, to confirm that a single virus particle is embedded; and
d) exposing the nucleic acid from the visualized, embedded single, viral particle and amplifying the exposed genomic viral nucleic acid in situ by multiple displacement amplification (MDA).

2. The method of claim 1, wherein
the solid matrix in step b) is low melting agarose in a well of a polytetrafluoroethylene (PTFE) slide.

3. The method of claim 1, wherein
the solid matrix in step b) is low melting agarose in a well of a 96-well dish.

4. The method of claim 1, wherein
in step b), the sorted sample is embedded in a thin layer of low melting agarose on a flat slide.

5. The method of claim 1, wherein the visualizing in step c) is performed by epifluorescent microscopy (EFM) and/or confocal microscopy.

6. The method of claim 1, wherein the nucleic acid in step d) is exposed by alkali lysis, or treatment with heat or a KOH solution.

7. The method of claim 1, further comprising, if more than one virus particle has been shown by the visualization in step c) to be embedded in the solid matrix, excising (etching out) from the solid matrix single virus particles, still embedded in the solid matrix, by laser capture dissection (LCM); exposing the nucleic acid from the excised single viral particles; and amplifying the exposed viral nucleic acid in situ by MDA.

8. The method of claim 7, wherein the nucleic acid of the excised, embedded, single virus particles is exposed by alkali lysis, or treatment with heat or a KOH solution.

9. The method of claim 1, further comprising sequencing the amplified viral nucleic acid.

10. The method of claim 7, further comprising sequencing the amplified viral nucleic acid.

11. The method of claim 9, further comprising confirming that the amplified nucleic acid is from a single virus particle and/or contains one or more viral nucleic acid sequences of interest.

12. The method of claim 11, wherein the confirmation is achieved by performing specific PCR, RAPD-PCR or Neural network training.

13. The method of claim 1, wherein the mixture of viral particles is from a natural population of viruses in an environmental sample.

14. The method of claim 1, wherein the mixture of viral particles is from a collection of clinical samples.

15. A method for characterizing the genotypes of viruses present in an environmental sample, comprising amplifying the nucleic acid of single particles of the viruses in the sample by a method of claim 1; confirming that the amplified nucleic acid is from a single virus particle by performing RAPD-PCR or Neural network training; and sequencing the amplified nucleic acid.

16. The method of claim 15, wherein the environmental sample comprises virioplankton communities in sea water.

17. A method for mining for useful genes in a sample containing a mixture of viruses, comprising amplifying the nucleic acid of single particles of the viruses by a method of claim 1 and identifying novel viral sequences and/or genes of interest from the amplified nucleic acid.

18. The method of claim 1, which is a high throughput method.

19. The method of claim 1, which is carried out without propagating or cultivating the virus particles in a host cell.

20. A kit for amplifying the genome from a single virus particle from a mixture of virus particles, comprising
reagents for subjecting the mixture of viral particles to flow cytometry, and
reagents for amplifying embedded nucleic acid by MDA and, optionally,
instructions for isolating single viral particles and amplifying their genomes, using the reagents in the kit.
